(19) United States
(12) Patent Application Publication
(10) Pub. No.: US 2024/0381868 A1
(43) Pub. Date: Nov. 21, 2024

(54) AGED AQUEOUS ANTIMICROBIAL COMPOSITION

(71) Applicant: EVONIK CORPORATION, Piscataway, NJ (US)

(72) Inventors: Weidong AN, Williamsville, NY (US); Ricky Mittiga, Fort Mill, SC (US); Elena Pisanova, Amherst, NY (US); John M. Rovison, JR., Sanborn, NY (US)

(73) Assignee: EVONIK CORPORATION, Piscataway, NJ (US)

(21) Appl. No.: 18/577,322

(22) PCT Filed: Jul. 7, 2022
(86) PCT No.: PCT/US2022/073515

§ 371 Date: Jan. 8, 2024

(30) Foreign Application Priority Data
Jul. 9, 2021 (EP) 21184635.7

(51) Int. Cl. A01N 37/36; A01P 1/00; A01P 3/00

(52) U.S. Cl. CPC A01N 37/36; A01P 1/00; A01P 3/00

(57) ABSTRACT

An aged aqueous antimicrobial composition is prepared by mixing a carboxylic acid selected from maleic acid, crotonic acid, malic acid, tartaric acid, malonic acid, citric acid and mixtures thereof, with hydrogen peroxide; reacting the mixture to form a percarboxylic acid; and ageing the mixture that has the percarboxylic acid for at least 14 days.

### FIELD OF THE INVENTION

The invention relates to a process for preparing an antimicrobial composition containing a peracid, to the antimicrobial composition prepared by this process and to the method of using the composition in antimicrobial treatment. The composition shows outstanding antimicrobial activity, generates little or no odor and can be safely used to decontaminate substances without degrading their organoleptic properties. Substances that may be treated include foods, such as meats, fruits, vegetables, and grains, beverages and packaging. The disclosed composition can also be used for any other antimicrobial treatment.

### BACKGROUND OF THE INVENTION

The processing of food products often leads to bacterial contamination both from the workers involved and from processing equipment, as well as from the food products themselves. Antimicrobial agents are therefore used during processing to reduce contamination, especially from potentially pathogenic bacteria such as E. coli and Salmonella.

Peracetic acid (PAA) is one of several biocides that have been approved for direct application to meat, poultry and other products. Unfortunately, due to the very low (0.15 ppm) limit for olfactory detection of PAA and acetic acid, these biocides are often unpleasant for workers to use. They also pose a safety hazard due to their volatility and tendency to irritate the eyes, lungs and skin of workers. In addition, PAA biocides may alter the organoleptic properties of food products (e.g., the texture, color, appearance or taste of meat proteins), thereby reducing product quality.

Various peracids have been investigated that could be used in the place of PAA for antimicrobial applications. These peracids are typically synthesized from hydrogen peroxide and organic acids and, in some instances, have been demonstrated to be highly effective against hard to kill microorganisms. For example, percitric acid (PCA) was shown to have a higher virucidal efficacy than PAA (P.Wutzler, et al., Ltrs. App. Microbiol. 39:194-198 (2004)), as well as high sporicidal efficacy (P. Wutzler, et al., J. Hosp. Infect. 79:75-76 (2005)). In addition, performic acid (PFA) and perpropionic acid (PPA) were found to be highly effective in the reduction of E. coli and enterococci in wastewater (Luukkonen, et al., Water Res. 85:275-285 (2015)).

Peracids are usually produced by the reaction of the corresponding carboxylic acids with hydrogen peroxide (HP) in the presence of sulfuric acid, which acts as a catalyst, as shown by the equation below:

$$R - COOH + {H}_{2} {O}_{2} ↔ R - COOOH + {H}_{2} O$$

The reaction is an equilibrium and is driven to the right by removal of water or using excess reagents.

There is a further need for peroxyacid-based antimicrobial compositions with little or no odor and high antimicrobial activities. Such compositions should reduce microbial load without negatively impacting worker safety or the environment and, at the same time, not alter the organoleptic properties of the food. In addition, the antimicrobial agent should be stable, environmentally compatible, and not generate toxic residues or byproducts.

### SUMMARY OF THE INVENTION

The present invention is based on the unexpected discovery that aging of aqueous compositions containing certain peracids results in a significant increase in their biocidal efficiency. Analysis of the aged compositions has shown that additional compounds of smaller molecules are naturally formed, presumably from the peracids or precursors of the peracids.

The obtained compositions have low or no odor and can be used for antimicrobial treatment of foods and beverages.

Such compositions allow for prolonged storage, transportation, and dilution of the concentrated compositions at the point of application provide high antimicrobial efficacy of the resulting diluted composition. Application of such diluted compositions facilitates the sanitizing of meat proteins and other foods without altering their organoleptic properties.

### DESCRIPTION OF THE INVENTION

### The Process for Preparing the Antimicrobial Composition

The invention provides a process for preparing an aqueous antimicrobial composition, comprising the steps:
a) mixing a carboxylic acid selected from maleic acid, crotonic acid, malic acid, tartaric acid, malonic acid, citric acid or mixtures thereof, hydrogen peroxide, water and an inorganic acid;
b) reacting the mixture obtained in step a) to form a percarboxylic acid derived from the carboxylic acid;
c) ageing the mixture comprising the percarboxylic acid obtained in step b) for at least 14 days, preferably for at least 16 days, more preferably for at least 18 days, more preferably for at least 20 days.

It is preferred that the ageing in step c) is carried out up to 11 months, up to 6 months, up to 5 months, up to 3 months, up to 107 days, up to 72 days, up to 51 days, or up to 36 days.

It is preferred that the ageing in step c) is carried out between 14 days and 11 months, between 14 days and 6 months, between 14 days and 5 months, between 14 days and 3 months, between 14 days and 107 days, between 14 days and 72 days, between 14 days and 51 days, or between 14 days and 36 days.

It is preferred that the ageing in step c) is carried out between 15 days and 11 months, between 15 days and 6 months, between 15 days and 5 months, between 15 days and 3 months, between 15 days and 107 days, between 15 days and 72 days, between 15 days and 51 days, or between 15 days and 36 days.

Surprisingly, it was found that aging certain peracid compositions results in better antimicrobial efficacy of the compositions. Without wishing to be bound by any particular theory, it is thought that this is due to additional compounds being naturally formed in the aged peracid compositions, presumably from the peracids or precursors of the peracids. Analysis of the aged compositions shows the existence of additional smaller molecules besides the peracid and its precursor.

The terms “organic acid”, “acid” and “carboxylic acid” are used interchangeably in the present invention. The terms “peracid”, “peroxyacid”, “peroxycarboxylic acid” and “percarboxylic acid” are analogous and are also used interchangeably in the present invention.

### The Carboxylic Acid

The carboxylic acid is selected from maleic acid, crotonic acid, malic acid, tartaric acid, malonic acid, citric acid or mixtures thereof. Of these, citric acid is particularly preferred. Surprisingly, the peracids derived from the these selected carboxylic acids have been found to undergo some ageing processes and to form the new compounds with higher antimicrobial activities than the original percarboxylic acids.

The aqueous mixture comprising the carboxylic acid combined with hydrogen peroxide forms peracid-peroxide solutions that generate non-detectable to minimum odor. At the same time, the formed peracids exhibit after ageing for at least 14 days a superior antimicrobial activity when compared to the freshly prepared compositions.

The selected carboxylic acids employed in the invention are generally suitable for the use in the food industry. Preferably, these carboxylic acids should be food grade or GRAS materials. Such carboxylic acids and the peracids derived thereof are particularly suitable in food processing. Some of the preferred carboxylic acids are commercially available as derivatives of the natural materials, for example, tartaric acid is derived from grapes and malic acid is commercially derived from apples.

### Additives

The reaction between the carboxylic acid and hydrogen peroxide can be accelerated using an inorganic acid, preferably a strong inorganic acid as a catalyst. This inorganic acid can preferably be selected from sulfuric acid, methane sulfonic acid, phosphoric acid, nitric acid, acid ion-exchange resin, and mixtures thereof. The use of such acids may facilitate achieving the equilibrium state in the obtained acid/peracid/hydrogen peroxide mixture.

A peroxide stabilizer is preferably added to at least one of the reactants or during at least one of the inventive process steps. All compounds generally suitable for stabilizing hydrogen peroxide and/or peracid solutions may be used as peroxide stabilizers in the invention. Such compounds are generally known from the prior art. Particularly preferably, commercially available food additives can be used in the invention, such as organophosphonates sold under the trade name DEQUEST® including, for example, 1-hydroxyethylidene-1,1-diphosphonic acid (DEQUEST® 2010); amin (tri(methylene-phosphonic acid)), (N[CH₂PO₃H₂]₃, DEQUEST® 2000); and/or ethylenediamine-[tetra(methylenephosphonic acid)] (DEQUEST® 2041). Other suitable stabilizers include pyridine derivatives, such as dipicolinic acid, pyridine tricarboxylic acid, and pyridine tetrcarboxylic acid. The weight ratio of the employed stabilizer to the employed carboxylic acid is usually less than 1:30, more preferably less than 1:50.

### Inventive Process Steps

The aqueous hydrogen peroxide solution, employed in step in the inventive process, may have a concentration of up to 100% by weight of H₂O₂, preferably at least 25% by weight, more preferably 30-75% by weight.

The initial concentration of hydrogen peroxide in the mixture obtained in step a) of the inventive process may range from 2 wt % to 60 wt % by weight, preferably 3 wt % to 40 wt %, more preferably 5 wt % to 30 wt %.

The molar ratio of the employed in the inventive process hydrogen peroxide to the employed carboxylic acid is usually from 0.5:1.0 to 4.0:1.0, preferably from 0.8:1.0 to 3.0:1.0, more preferably from 1.0:1.0 to 2.5:1.0.

The initial concentration of the carboxylic acid in the mixture obtained in step a) of the inventive process is preferably at least 20% by weight, more preferably in the range 20-50 wt %.

Step a) of the inventive process may comprise mixing:
a) 20-50 wt %, preferably 25-40 wt % of the carboxylic acid;
b) 5-40 wt %, preferably 12-30 wt % of hydrogen peroxide, calculated as 100% H₂O₂;
c) 0.2-5 wt %, preferably 0.5-3 wt % of the inorganic acid;
d) optionally 0.1-5 wt %, preferably 0.2-2 wt % of a stabilizer;
e) water (balance to 100%).

It was found that an excess of water may degrade the peracid in diluted compositions and that minimizing amounts of water used in steps a) and b) of the process according to the invention usually enhances stability of the obtained peracid mixtures. Thus, the amount of water in the mixture obtained in step b) is preferably less than 60 wt %, more preferably less than 40 wt %.

In step b) of the inventive process, the carboxylic acid and hydrogen peroxide are preferably reacted until achieving the concentration of the peracid close to its equilibrium concentration.

The term “close to the equilibrium concentration” means that the concentration of the percarboxylic acid in the mixture deviate by at most 20%, preferably by at most 10% from the maximal concentration thereof achievable in this mixture.

The peracid concentration close to its equilibrium concentration is preferably achieved in step b) of the process prior to ageing of the mixture in step c). However, it is not necessary. The mixture with a lower than equilibrium concentration of the peracid may be aged in step c) of the process resulting in antimicrobial compositions with an antimicrobial performance superior to those of the non-aged peracid compositions.

The rate of achieving the equilibrium in the inventive process usually depends on the temperature and the concentrations of the reactants in the mixture.

Step b) of the process of the invention is preferably carried out at 0° C. to 50° C., more preferably at 4° C. to 30° C., more preferably at 10° C. to 25° C.

The duration of step b) of the inventive process may be from 1 minute to 8 weeks, preferably from 10 minutes to 4 weeks, more preferably from 1 hour to 2 weeks.

The mixture obtained in step b) or c) of the process of the invention can be diluted with water to contain from 50 ppm to 10,000 ppm of the percarboxylic acid.

Ageing of the mixture in step c) of the inventive process can be carried out at the temperature 0° C. to 50° C., preferably 4° C. to 40° C., more preferably 15° C. to 30° C., more preferably at 20 to 25° C., particularly preferably at ambient temperature of 25° C.

### The Aqueous Antimicrobial Composition

A further object of the invention is an aqueous antimicrobial composition obtained by the inventive process.

The molar ratio of the (carboxylic acid):(percarboxylic acid):(hydrogen peroxide) in the inventive composition is preferably (0.5-50.0):(1.0):(1.0-30.0), more preferably (1.0-10.0):(1.0):(3.0-15.0). This ratio ensures the optimum balance of biocidal efficacy of the antimicrobial composition and cost.

The absolute concentrations of the components of the composition may vary depending on the dilution extent thereof and on the intended application.

The content of the percarboxylic acid in the composition is usually in the range 10 ppm to 40 wt %. The content of the percarboxylic acid in the concentrated composition according to the invention may be from 0.5 wt % to 40 wt %, preferably 2 wt % to 30 wt %. The content of the percarboxylic acid in the diluted composition of the invention may be from 10 ppm to 20,000 ppm, preferably 50 ppm to 5,000 ppm.

The aqueous antimicrobial composition according to the invention is typically characterized by a high stability during the storage, especially when cooled. The high stability, e.g., increased shelf life, means that the stable peracid compositions retain a relatively high level of peracid over a given period of time. In this regard, the concentration of the peracid after one month of storage at +4° C. should be no lower than 60%, preferably at least 80%, more preferably at least 85% of the maximum concentration thereof. After three months of storage at −4° C. the concentration of peracids should be no lower than 90% of the maximum concentration thereof.

The inventive composition obtained after aging of the mixture comprising a peracid, an acid and hydrogen peroxide differs from the freshly prepared mixture. Thus, such an aged composition usually comprises other than peracid and acid organic compounds, which can be identified by mass-spectrum (MS) analysis of the mixture. Thus, MS spectrum of a freshly prepared percitric acid contains typical peaks of citric and percitric acids with M/z=207, 191, 173, 111, 87. Mass spectrum of an aged percitric acid additionally shows the peaks with M/z=57, 59, 89, 101, 103, 145 and 195. This confirms formation of some new products, presumably including 3-oxoglutaric acid, during the ageing process. Formation of the new products has also been confirmed in the other selected aged carboxylic acid/percarboxylic acid mixtures.

The inventive composition is preferably obtained using citric acid as the carboxylic acid and shows peaks with M/z=57, 59, 89, 101, 103, 145 and 195 in the mass spectrum analysis of the composition obtained by liquid chromatography with mass spectrometry (HPLC-MS).

### Method of Use of the Aqueous Antimicrobial Composition

The invention further provides a method of use of the inventive antimicrobial composition for an antimicrobial treatment, especially of a food product, a hard surface or an aseptically packed beverage, of water used for washing or processing thereof, as well as in areas including food plants, kitchens, bathrooms, factories, hospitals, and dental offices.

The composition of the present invention is a particularly effective antimicrobial agent. While not wishing to be bound by any theory of operation, it appears that the molecules obtained by degradation of the percarboxylic acid present in the aged composition have better antimicrobial activity than the percarboxylic acid itself.

The suitable food product may be selected from a vegetable, fruit or grain, meat protein, red meat such as beef, poultry, seafood.

The suitable hard surface may be selected from a cutting board, sink, cutting blade, conveyor, picker, bird washer, on-line or off-line processing equipment, a carcass, hide or flume.

The antimicrobial treatment with the inventive composition carried out for at most 1 minute, more preferably for at most 30 seconds at 20-25° C. is usually sufficient to reduce the microbial contamination by at least 50%, preferably by at least 70%, more preferably by at least 90%.

The duration of the antimicrobial treatment is preferably chosen so that the microbial contamination is reduced by at least 50%, preferably by at least 70%, more preferably by at least 90%.

The described antimicrobial treatment is preferably effective at killing one or more of the food-borne pathogenic bacteria associated with a food product, including, but not limited to Salmonella typhimurium, Salmonella javiana, Campylobacter jejuni, Listeria monocytogenes, and Escherichia coli, as well as yeasts, molds, and spores.

The compositions of the present invention can kill a wide variety of microorganisms on a surface of food processing equipment, on the surface of a food product, in water used for washing or processing of food product, on a health care surface, or in a health care environment. The inventive composition has been found to provide optimal antimicrobial efficacy and stability in the presence of high organic loads.

The inventive antimicrobial composition may be provided as a concentrate for mixing with water at the point of use. Normally, the antimicrobial compositions of the present invention will be employed using conventional equipment and at a temperature of approximately 0-70° C. The composition should preferably be diluted to 50-10,000 ppm of active peracid concentration before use and contact with the foods or surfaces of food processing equipment or other contaminated surfaces should usually be maintained for not less than five seconds.

The diluted composition can be applied by washing, dipping, spraying and other standard methods used in meat processing. The diluted compositions can be applied in batch or continuous processes and are suitable for use in automated processes, thereby reducing cost for the food processing plant and improving the safety for workers.

### BRIEF DESCRIPTION OF THE DRAWINGS

FIG. 1 is a chromatograph obtained by performing HPLC on a fresh and aged percitric acid compositions PCA-1 as described in Example 4.

FIG. 2 shows the results obtained by performing mass spectroscopy on an aged percitric acid composition PCA-1 composition as described in Example 4.

### EXAMPLES

The following examples are presented to offer further illustration of the present invention but are not intended to limit the scope of the invention in any manner whatsoever.

### Preparation of Test Solutions

Solutions of peracids were prepared by dissolving the appropriate weight of the organic acids in the aqueous hydrogen peroxide (70% solution from PeroxyChem LLC). De-ionized water (DI water) was then added to obtain the desired concentrations. All the solutions were clear and homogeneous when initially prepared and remained so for the duration of the experiment.

The concentrations of formed peracids and hydrogen peroxide were monitored by titration on an auto-titrator and by using Chemetrics test kits K7913F and K-5543.

### Example 1

Two solutions were prepared using solid citric acid (47.9 wt %), 70 wt % hydrogen peroxide (21.8 wt %) and the rest DI water. 0.6 wt % Dequest 2010 stabilizer was added to both solutions. To the first solution (PCA-1), 4.9 wt % concentrated sulfuric acid was added as a catalyst. To the second solution (PCA-2), 0.6 wt % of sulfuric acid was added. Both solutions were kept at room temperature and periodically tested for the concentration of the components using an auto-titrator and standard titration methods. Concentrations of the components are shown in the Table 1.

TABLE 1
Formation and Stability of Peroxycitric acid

| | | Concentration, Moles/kg | | | | | | |
|---|---|---|---|---|---|---|---|---|
| Composition | Component | 1 day | 3 days | 7 days | 14 days | 22 days | 32 days | 46 days |
| PCA-1 | Peracid | 1.063 | 1.269 | 1.082 | 0.832 | 0.601 | 0.409 | 0.221 |
| | H₂O₂ | 3.647 | 3.176 | 2.765 | 2.176 | 1.647 | 1.118 | 0.647 |
| PCA-2 | Peracid | 0.337 | 0.668 | 0.822 | 0.707 | 0.644 | 0.490 | 0.365 |
| | H₂O₂ | 4.265 | 3.941 | 3.500 | 3.029 | 2.706 | 2.206 | 1.735 |

Percitric acid (PCA) is formed by a reaction of citric acid with hydrogen peroxide in the presence of H₂SO₄ catalyst. As shown in Table 1, maximum concentrations of PCA were reached after 3-7 days. The maximum concentration was higher with a higher percent of the catalyst. However, in both cases, decomposition of PCA started after reaching a maximum, so that in a month concentration of both PCA and hydrogen peroxide decrease substantially. The data shown in the Table 1 demonstrates instability of PCA over time at ambient temperature.

The freshly made PCA solutions had no smell; however, surprisingly a pleasant fruity smell had developed over time while the PCA went through the aging process. This unexpected finding could provide PCA an appealing advantage over other peracids, e.g. PAA-based formulations that generate a pungent smell and have industrial hygiene concerns.

### Example 2

Various other peracids were also prepared using corresponding organic acid precursors and hydrogen peroxide as described in the Example 1. Only the acids with low or no odor were used in the experiment. The solutions were kept at room temperature and periodically tested for the concentration of the components using an auto-titrator and standard titration methods. Concentrations of the formed peracids are shown in the Table 2.

As can be seen from the data in the Table 2, the maximum concentration and stability of peracids varies significantly. Thus, peroxalic acid was formed in very low concentration (<1.0 wt %), and permalonic acid decomposed quickly after formation.

The highest yield was found in the case of glutaric acid that formed relatively stable perglutaric acid. Permaleic acid, although formed at max 5.1 wt %, was stable for at least 78 days.

TABLE 2
Formation and Stability of Peracids

| | Peracid Concentration, Moles/kg | | | | | |
|---|---|---|---|---|---|---|
| Peracid | 1 day | 3 days | 7 days | 14 days | 22 days | 78 days |
| Peroxymalonic | — | 0.442 | 0.200 | 0.083 | — | — |
| Peroxyoxalic | — | 0.047 | 0.066 | 0.057 | — | — |
| Peroxymaleic | 0.235 | — | 0.386 | 0.379 | 0.348 | 0.318 |
| Peroxyglutaric | 0.993 | 1.189 | 1.081 | 0.986 | 0.919 | — |

### Example 3

The effect of temperature on the stability of peracid solutions was evaluated. Percitric acid (PCA-1) was prepared as described in Example 1, but after preparation one part of the solution was kept in refrigerator at 4° C., and the second part in a freezer at −4° C. Periodically, the compositions were tested for the concentration of the components. The results are shown in the Table 3.

TABLE 3
Formation and Stability of Peroxycitric acid

| Storage | Peracid Concentration, Moles/kg | | | | | | |
|---|---|---|---|---|---|---|---|
| temperature, | 3 | 15 | 36 | 51 | 72 | 107 | 128 |
| ° C. | days | days | days | days | days | days | days |
| +4 | 1.192 | 1.385 | 1.337 | 1.303 | 1.192 | 1.067 | — |
| −4 | 1.260 | 1.327 | 1.409 | 1.486 | 1.418 | 1.481 | 1.505 |
| Room | 1.269 | 0.832 | 0.346 | 0.188 | 0.087 | — | — |

The data in the Table 3 shows the effect of temperature on the peracid stability. At room temperature, the PCA-1 starts decomposing after 1 week of storage, whereas freezing improves the stability. In fact, the concentration of PCA continues increasing when the composition is kept at −4° C. Therefore, freezing the peracid solution can be used as an efficient way to prolong its storage time.

### Example 4

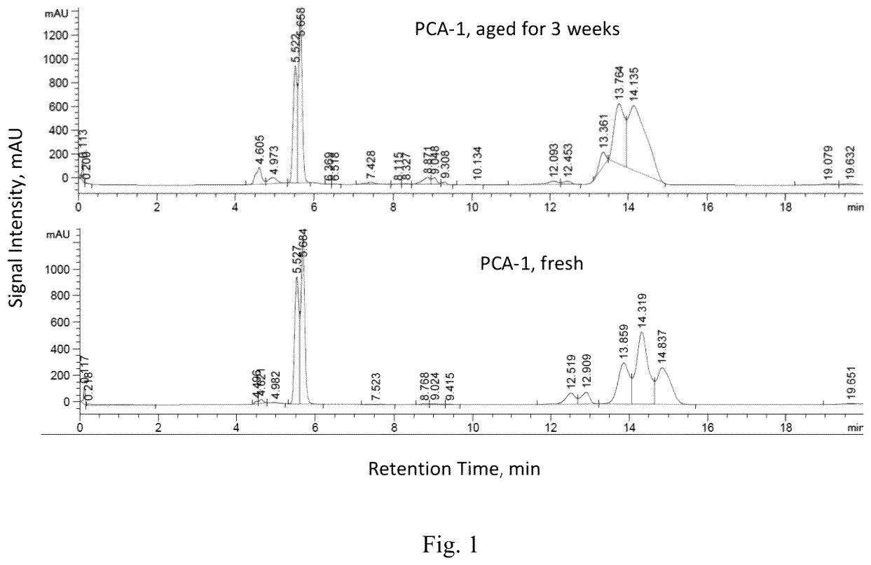

Aged and fresh peracid compositions were tested using the Liquid chromatography-mass spectrometry (HPLC-MS) analytical technique. The results are shown in FIG. 1. The chromatograms of aged compositions have shown unexpected differences in comparison with the fresh compositions, and the differences increase with the time of aging. Thus, fresh PCA-1 composition showed the following peaks: 5.6 min (H₂O₂), citric acid (14.5 min) and percitric acid (14.0 min). MS spectrum of citric acid contained typical peaks of citric and percitric acids (M/z=207, 191, 173, 111, 87). (https://www.researchgate.net/figure/MS-MS-SPECTRA-OF-CITRIC-ACID-COMMERCIAL-STANDARD-AND-COMPOUND-4-m-z-1911111-MS-MS_tbl3_236590427).

After aging, several small new peaks appeared at HPLC chromatogram of PCA-1 composition, in particular, single peaks at 5.0 and 7.3 min, and double peaks in the areas of 8.5-9.5 min and 11.8-12.8 min. Double peaks could be caused by an acid-peracid couple, similar to a double peak of citric-percitric acid at 14.0-14.5 min, as described in (Liquid Chromatographic Separation and Simultaneous Analyses of Peroxycitric Acid and Citric Acid Coexisting with Hydrogen Peroxide in the Equilibrium Mixture. Islam M., Ferdousi B., et al. Journal of Chromatographic Science, Vol. 49, January 2011).

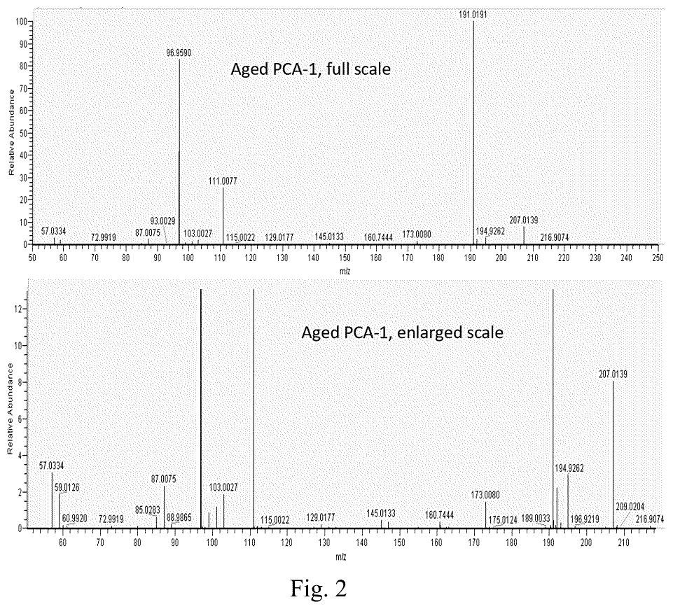

The observed changes in the mass spectra of aged compositions can be also attributed to the natural formation of small amounts of new compounds in the PCA compositions. In particular, new small peaks were observed in the PCA-1 composition after aging with M/z=57, 59, 89, 101, 103, 145 and 195, as shown in FIG. 2. These changes indicate a likely slow natural oxidation in the system citric acid/H₂O₂/percitric acid. For example, it is known that citric acid can be oxidized with hydrogen peroxide to 3-oxoglutaric acid that has characteristic peaks at M/z=145, 103, 57, and 59 (see http://www.hmdb.ca/spectra/ms_ms/129039).

These results show that the aging acid/H₂O₂/peracid systems results in slow formation of the products of internal oxidation. Besides the above-mentioned unexpected development of a pleasant fruity smell, such aged compositions have also demonstrated surprisingly improved stability and antimicrobial efficacy as shown in some of the following examples.

### Example 5

In this test, the role of aging time on the efficacy of the composition was investigated. PCA-1 composition was tested immediately after the maximum peracid concentration was achieved, and then after aging at 25° C. for 1, 3, and 4 weeks respectively. All compositions were diluted to the same PCA concentration of 4.81×10⁻⁴ moles/kg. The effect of peracids on microbial viability was assayed using a Salmonella suspension testing method as described below. Results are shown in the Table 4.

The effect of peracids on microbial viability was assayed using a suspension testing method as described below.

Inoculum Preparation. Salmonella enterica ATCC 14028 or E. coli 8739, as model Gram-negative bacteria, were grown in trypticase soy broth for approximately 24 hours at 35° C. Then the suspension was diluted at a ratio 1:10 with Butterfield's buffer. This diluted suspension was added to sterile fetal bovine serum at a ratio 1:1 in order to produce a working inoculum containing 50% organic load. The further dilution of the working inoculum into the test matrix at a 1:10 ratio resulted in a 5% organic load.

Test solutions. The peracid solutions tested were prepared by diluting the concentrated solutions with deionized water to the desired concentration. The concentrates were freshly prepared and used right after the maximum concentration was reached.

Test method. 9 mL aliquots of the test solutions were added to a sterile centrifuge tube using sterile disposable serological pipettes. Then, a 1 mL aliquot of working inoculum was added to the test solution and vortexed briefly to mix. A 1 mL aliquot was removed from the mixture at specific time points following inoculation, and immediately added to a 9 mL volume of Letheen neutralizing broth containing 0.5% sodium thiosulfate. This broth was then shaken to mix, sonicated for 5 minutes, vortexed for 30 seconds, diluted serially into Butterfield's buffer, and the dilutions plated on 3M™ Petrifilm™ Aerobic Count Plates (APC). The plates were incubated for 48 hours at 35° C., and then counted manually. Log 10 calculations were performed to obtain the Log 10 CFU/mL of the solutions at each time point.

The data in the Table 4 show that aging the PCA-1 composition surprisingly resulted in a significantly improved efficacy against Salmonella. Freshly prepared PCA-1 composition at 4.81×10⁻⁴ moles/kg had only 2.9 Log ₁₀ reductions in 30 minutes treatment; however, upon aging for 1, 3, and 4 weeks, the reductions at the same 4.81×10⁻⁴ moles/kg increased to 3.8 Log ₁₀ at 30 minutes, total kill at 15 minutes, and total kill at 15 minutes respectively.

TABLE 4
Antimicrobial efficacy with Aged PCA-1 Formulations
at 4.81 × 10⁻⁴ moles/kg

| Time Aged, | Treatment | Log₁₀ | Log₁₀ |
|---|---|---|---|
| weeks | Time, min | control | reduction |
| 0 | 5 | 6.5 | 0.7 |
| 0 | 15 | 6.5 | 2.4 |
| 0 | 30 | 6.5 | 2.9 |
| 1 | 5 | 6.5 | 1.0 |
| 1 | 15 | 6.5 | 2.6 |
| 1 | 30 | 6.5 | 3.8 |
| 3 | 5 | 6.5 | 4.0 |
| 3 | 15 | 6.5 | Total kill |
| 4 | 5 | 6.5 | 4.6 |
| 4 | 15 | 6.5 | Total kill |

### Example 6

In this test, the role of aging time on the efficacy of permalic acid (PmA) was investigated. The equilibrated composition contained 0.313 moles/kg PmA and 3.794 moles/kg H₂O₂. The composition was tested immediately after the maximum peracid concentration was achieved, and then after 2 weeks ageing at 25° C. Both compositions were diluted to the same PmA concentration of 6.67×10⁻⁴ moles/kg. The effect of peracids on microbial viability was assayed using a Salmonella suspension testing method as described in the Example 5. Results are shown in the Table 5.

The data in the Table 5 show that aging the PmA composition results in an improved efficacy against Salmonella similar to that of the percitric based compositions.

TABLE 5
Antimicrobial efficacy with Aged PmA Formulations
at 6.67 × 10⁻⁴ moles/kg

| Time Aged, | Treatment | Log₁₀ | Log₁₀ |
|---|---|---|---|
| weeks | Time, min | control | reduction |
| 0 | 1 | 6.5 | 1.7 |
| 0 | 3 | 6.5 | 2.7 |
| 0 | 5 | 6.5 | 3.5 |
| 2 | 3 | 6.5 | 3.5 |
| 2 | 5 | 6.5 | Total kill |

1. A process for preparing an aqueous antimicrobial composition, the process comprising:
a) mixing a carboxylic acid selected from the group consisting of maleic acid, crotonic acid, malic acid, tartaric acid, malonic acid, citric acid, and mixtures thereof, hydrogen peroxide, water and an inorganic acid to obtain a first mixture:
b) reacting the first mixture obtained in a) to form a percarboxylic acid derived from the carboxylic acid to obtain a second mixture;
c) ageing the second mixture comprising the percarboxylic acid obtained in b) for at least 14 days, wherein the ageing of the second mixture is carried out at a temperature of from 0° C. to 50° C. to obtain a third mixture.

2. The process according to claim 1, wherein a duration of b) is selected so that a concentration of the percarboxylic acid in the second mixture deviates by at most 20% from a maximal concentration thereof achievable in the second mixture.

3. The process according to claim 1, wherein the ageing of the second mixture in c) is carried out at a temperature of 15° C. to 50° C.

4. The process according to claim 1, wherein the inorganic acid is selected from the group consisting of sulfuric acid, methane sulfonic acid, phosphoric acid, nitric acid, an acid ion-exchange resin, and mixtures thereof.

5. The process according to claim 1, wherein an employed aqueous hydrogen peroxide solution has a concentration of at least 25% by weight of H₂O₂.

6. The process according to claim 1, wherein a molar ratio of the employed hydrogen peroxide to the employed carboxylic acid is from 0.5:1.0 to 4.0:1.0.

7. The process according to claim 1, wherein an initial concentration of the carboxylic acid in the first mixture obtained in a) is at least 20% by weight.

8. The process according to claim 1, wherein the second mixture obtained in b) or the third mixture obtained in c) is diluted with water to contain from 50 ppm to 10,000 ppm of the percarboxylic acid.

9. The aqueous antimicrobial composition obtained by the process of claim 1.

10. The composition according to claim 9, wherein the carboxylic acid is citric acid, and the aqueous composition shows peaks with M/z=57, 59, 89, 101, 103, 145 and 195 in a the mass spectrum analysis of the composition obtained by liquid chromatography with mass spectrometry (HPLC-MS).

11. A method for antimicrobial treatment, the method comprising:
applying the composition according to claim 9 to a substrate.

12. The method according to claim 11, wherein the substrate is a food product, wherein the food product is at least one selected from a vegetable, fruit, grain, meat protein, red meat, poultry, and seafood.

13. The method according to claim 11, wherein the substrate is a hard surface selected from the group consisting of a cutting board, a sink, a cutting blade, a conveyor, a picker, a bird washer, an on-line or off-line processing equipment, a carcass, a hide, and a flume.

14. The method according to claim 11, wherein the antimicrobial treatment is carried out for at most 1 minute at 20-25° C. to reduce a microbial contamination by at least 50%.

15. The method according to claim 11, wherein the antimicrobial treatment is effective at killing one or more food-borne pathogenic bacteria associated with a food product as well as yeasts, molds, and spores.

16. The process according to claim 3, wherein the ageing of the second mixture in c) is carried out at an ambient temperature of 25° C.

17. The process according to claim 5, wherein the employed aqueous hydrogen peroxide solution has a concentration of at least 30-75% by weight H₂O₂.

18. The process according to claim 6, wherein the molar ratio of the employed hydrogen peroxide to the employed carboxylic acid is from 0.8:1.0 to 3.0:1.0.

19. The method according to claim 11, wherein the substrate is at least one selected from the group consisting of an aseptically packed beverage, water used for washing or processing thereof, and areas including food plants, kitchens, bathrooms, factories, hospitals, and dental offices.

20. The method according to claim 15, wherein the antimicrobial treatment is effective at killing at least one or more of food-borne pathogenic bacteria selected from the group consisting of Salmonella typhimurium, Salmonella javiana, Campylobacter jejuni, Listeria monocytogenes, and Escherichia coli.
